(19) United States
(12) Patent Application Publication
(10) Pub. No.: US 2009/0123395 A1
(43) Pub. Date: May 14, 2009

(54) Flavour Compositions

(76) Inventors: John Martin Behan, Kent (GB); David Jonathan Bradshaw, Kent (GB); Jonathan Richards, Kent (GB); Michael John Munroe, Kent (GB)

Correspondence Address: MORGAN LEWIS & BOCKIUS LLP, 1111 PENNSYLVANIA AVENUE NW, WASHINGTON, DC, 20004, US

(21) Appl. No.: 12/330,624

(22) Filed: Dec. 9, 2008

Related U.S. Application Data
(62) Division of application No. 10/545,746, filed on Aug. 17, 2005.

(30) Foreign Application Priority Data
Feb. 18, 2003 (GB) 0303676.1

(51) Int. Cl. A61K 8/21; A61K 8/34; A61K 8/97; A61P 1/02

(52) U.S. Cl. 424/52; 424/49; 424/58

(57) ABSTRACT

Flavor compositions which reduce or prevent dental cavities based on the use of flavor materials found to inhibit the production of acid by micro-organisms in the oral cavity, e.g. lactic acid production from glucose by Streptococcus mutans. The compositions include at least two flavor materials from group (a) and at least one flavor material from group (b). Representative group (a) flavors are decanol, nonanol and cinnamic aldehyde while typical group (b) flavors are nonanal, citral and peppermint oil of material origin.

### FIELD OF THE INVENTION

This invention relates to flavour compositions, i.e. a mixture of flavour materials, to products containing such flavour compositions, and to the use of a flavour material or flavour composition for inhibiting or reducing acid-producing oral bacteria, particularly the bacterium Streptococcus mutans, implicated in initiating dental caries (tooth decay).

### BACKGROUND TO THE INVENTION

Dental plaque is a soft whitish material which forms on the surfaces of the teeth. Plaque is a matrix of bacteria, bacterial products and salivary and other host-derived components. One of the bacteria present in dental plaque is Streptococcus mutans, which converts dietary sugar into dextran, an insoluble, inert gelatinous polysaccharide which enables the bacterium to adhere to the tooth surface.

The excessive and/or frequent consumption of fermentable dietary sugars can lead to the enrichment of particular groups of bacteria, especially Streptococcus mutans, in dental plaque. Dental caries results from the dissolution of tooth enamel (demineralisation) by organic acids generated by bacteria. Streptococcus mutans is particularly important in the formation of dental caries because the bacterium rapidly generates large quantities of lactic acid from dietary sugars, whilst concomitantly displaying unusual acid tolerance and tenacity to tooth surfaces.

A number of strategies are employed to combat the development of dental caries.

One approach includes mechanical oral hygiene measures, e.g. brushing, to physically remove plaque. However, brushing alone is insufficient to remove all plaque that may form on the teeth or to prevent the formation of further plaque.

Numerous oral care compositions are known which include a variety of antimicrobial compounds, such as sodium dodecyl sulphate, essential oils, and other miscellaneous agents for inhibiting the development of dental caries. For example, WO 98/44901 concerns oral hygiene compositions including an antimicrobial agent selected from cedarwood oil, chloramphenicol, citronella oil, Glycyrrhiza glabra extract, juicy fruit basil oil, lemon basil oil, and Rosmarinus officinalis oil; EP 0819380 describes p-oxybenzoic acid esters such as methyl, ethyl and butyl p-oxybenzoates which possess bacteriocidal, antibacterial or bacteriostatic activity against Streptococcus mutans; U.S. Pat. No. 4,661,342 describes oral compositions containing hydroxamic acids such as 2-(4-butoxyphenyl)acetohydroxamic acid to prevent Streptococcus mutans from colonising in the oral cavity; U.S. Pat. No. 4,590,215 discloses that 1-alpha-cadinol inhibits Streptococcus mutans growth, polyunsaturated long-chain alcohols such as linolenyl alcohol and linoleyl alcohol are also described for use to this effect in U.S. Pat. No. 4,372,978; DE 4221103 discloses mixtures of myrrh extract or oil, mulberry bark extract, Cimicifuga heraleifolia extract and green tea extract, as having antibacterial activity against cariogenic bacteria. A number of these antimicrobial strategies will also inhibit the generation of acid from fermentable dietary sugars.

A further strategy for combating the development of dental caries is the replacement of conventional, readily fermentable dietary sugars in consumable products with non-fermentable ingredients, e.g. sweeteners, weakly fermentable sugars, or sugars, which interfere with normal sugar metabolism. For example, U.S. Pat. No. 5,294,449 discloses the use of erythrose in chewing gum to deliver anti-caries properties; GB 2046757 discloses the use of aldosylfructoside in this way; and EP 0438912 describes an edible composition which includes a bulking agent comprising polydextrose, an encapsulated flavouring agent and an effective amount of an intense sweetening agent.

Still other approaches interfere with the formation of the polysaccharide dextran in order to reduce the adherence of cariogenic bacteria to teeth or plaque. For example, EP 0704202 discloses cycloisomaltooligosaccharide as an active which inhibits glucan synthetase from Streptococcus mutans; and U.S. Pat. No. 4,912,089 discloses inhibition of glucan production by Streptococcus mutans using a purified Gymnemic acid derived from Gymnema sylvestre.

One of the most important caries preventive measures known is the use of fluoride which may be delivered, e.g. via consumer products such as oral care products or via fluoridation of public drinking water supplies. Drinking water is usually fluoridated using sodium fluoride, whereas, in the case of oral care products, fluoride may be incorporated in the form of a variety of salts including sodium salts, e.g. sodium fluoride and sodium monofluorophosphate, strontium salts, calcium salts etc. Fluoride typically acts to prevent caries by its incorporation into dental enamel. Such incorporation renders dental enamel less susceptible to demineralisation, whilst also promoting remineralisation. Fluoride is also known to inhibit a, variety of bacterial metabolic processes, in particular, carbohydrate metabolism. Several documents also disclose the combination of fluoride with other agents. For example, US 2002068039 discloses the use of a grapefruit seed extract in synergistic combination with a fluoride ion-providing compound to inhibit the growth and metabolism of, and to kill, plaque bacteria.

### SUMMARY OF THE INVENTION

The present invention is based on extensive testing of flavour materials to determine whether a particular material is capable of inhibiting the production of acid from the metabolism of dietary sugar by micro-organisms present in the oral cavity, and more particularly inhibiting lactic acid production from glucose by Streptococcus mutans. Based on this testing, flavour materials were identified, which whilst known, may possess hitherto unappreciated properties in terms of inhibiting or reducing acid-producing bacteria. The invention thus enables flavour compositions to be defined that reduce or prevent dental caries. Additionally, in a preferred embodiment, the invention enables flavour compositions to be formulated comprising flavour material(s) which selectively target and inactivate the acid-producing bacteria whilst preserving the remaining protective oral cavity microflora.

Accordingly, in one aspect, the present invention provides a flavour composition comprising at least two flavour materials selected from the following Group (a) materials: decanol, nonanol, decanal, anethole synthetic, cardamom oil, cinnamic aldehyde, ionone alpha, origanum, tarragon, thymol; and at least one flavour material selected from the following Group (b) materials: nonanal, Aniseed rectified, basil oil, camomile oil, citral, clove bud oil, Damascone F, ginger, Tea Tree Pure, peppermint oil of natural origin.

Cardamom oil is conveniently cardamom English.

Cinnamic aldehyde is conveniently cinnamic aldehyde extra, available from Quest International.

Basil oil is conveniently basil comores.

Camomile oil is conveniently camomile English.

Clove bud oil is preferably rectified, e.g. clove bud rectified extra.

For enhanced inhibition of acid-producing bacteria, preferably, flavour compositions of the invention comprise at least three flavour materials from Group (a).

Also preferred are flavour compositions comprising at least two flavour materials from Group (b).

Flavour compositions in accordance with the invention preferably comprise at least 3% by weight, more preferably at least 6% by weight and even more preferably at least 10% by weight, of flavour materials from Group (a); and preferably at least 3% by weight, more preferably at least 10% by weight and even more preferably at least 25% by weight, of flavour materials from Group (b).

Conveniently, Group (a) and Group (b) flavour materials may together comprise at least 6% by weight of the total weight of the flavour composition, preferably at least 15% by weight, more preferably at least 30% by weight, even more preferably at least 40% by weight and most preferably at least 50% by weight.

Peppermint oil useful herein is of natural origin. Preferably, the peppermint oil is a Piperita type from the far west regions of the United States, e.g. Peppermint American Rectified, Peppermint American Yakima Rectified, Peppermint American Willamette Natural, which is preferably rectified. Also preferred for use in a composition of the invention is an Arvensis type peppermint oil, typically having a total terpene content of less than 3.2% by weight, e.g. Peppermint Indian Rectified, Peppermint Arvensis Terpeneless ACF153, Peppermint Chinese Triple Rectified (available from Quest International).

The ingredients of the composition are known flavour materials which are readily available commercially in grades suitable for various intended purposes. Details of the flavour materials and potential suppliers thereof are mentioned, for example, in “Allured's Flavor and Fragrance Materials 2002”, Allured Publishing Corp., Carol Stream, Ill., USA, ISBN 0-931710-84-7.

Also included within the scope of the invention is a method, particularly a cosmetic method, for reducing or preventing acid-producing bacteria by introducing in the oral cavity a flavour composition in accordance with the invention.

The flavour materials useful in a flavour composition of the invention are capable of inhibiting the production of acid by micro-organisms present in the oral cavity. In particular, the flavour materials are capable of inhibiting the production of lactic acid from glucose by the bacterium Streptococcus mutans present in the oral cavity.

One property that characterises the effectiveness of a compound, e.g. a flavour material, to inhibit the production of acid by the micro-organism Streptococcus mutans in the oral cavity, is the minimum inhibitory concentration, or MIC, of the compound. The MIC is the minimum amount of a compound (e.g. in ppm) at which no bacterial growth is observed. Generally, the lower the MIC of a compound for a bacterium, the more effective the compound will be at inhibiting bacterial growth. At concentrations above the MIC, a compound may act by directly killing existing viable bacteria or inhibiting the growth and reproduction of the bacteria (antimicrobial effect). At concentrations below the MIC, a compound may interfere with the metabolic process, e.g. by inactivating the bacteria producing acid, but typically does not inhibit the growth and reproduction of bacteria (sub-lethal or sub-MIC effect).

The inhibitory effect of a flavour composition comprising the flavour materials useful herein can be achieved antimicrobially, or more surprisingly, sub-lethally.

The antimicrobial effects of compounds, e.g. flavour materials, are usually divided into two types; they can either inhibit bacterial growth (bacteriostatic action) or alternatively they can act by directly killing existing viable bacteria (bactericidal action).

The bacteriostatic action of a compound “X” (such as a flavour material) against a particular bacterium, can be tested for in vitro by inoculating a standard, small number of bacteria into broths containing an appropriate range of concentrations of X. The broths are then incubated for a suitable time, and growth compared with a control containing no inhibitor. The broth containing the lowest concentration of X which shows reduction of growth compared to the control broth, is defined as the minimum inhibitory concentration (MIC).

The determination of the bactericidal action of a compound “Y” (such as a flavour material) is carried out by adding various concentrations of compound Y to replicate broths containing relatively high, standard numbers of bacteria. After a certain period allowing any antibacterial activity to take place, aliquots of the bacterial cultures are diluted (usually in 10-fold steps) and dispensed onto agar plates. The plates are incubated with the expectation that each viable cell should produce a visible colony. The numbers of colonies are multiplied to take account of the dilution, to establish the number of viable cells in the broths. Once again, the broths containing compound Y are compared with an untreated control broth. The minimum concentration of compound Y which causes a reduction in the viable number of bacteria is the minimum bactericidal concentration (MBC). MBC can also be expressed in terms of the MBC required to produce a certain degree of killing (for example, a 3 log₁₀ reduction in count, equivalent to a 99.9% kill). Still further, the MBC can be expressed in kinetic terms—the time of exposure to an agent required for a given MBC effect.

A further possibility is that the process of inhibition could be sub-lethal (or sub-MIC), whereby the flavour materials interfere with the metabolic process, but typically do not inhibit bacterial growth.

Three modes of inhibiting the production of lactic acid are possible. In the first mode, the flavour materials (or flavour compositions) may act by direct (overt antimicrobial) killing of oral cavity bacteria, e.g. by more than 10-fold; in the second mode, they may inhibit acid generation whilst maintaining a microbial cell viability of at least 70%; in the third mode, they may inhibit acid generation at a concentration below the minimum inhibitory concentration (MIC), determined as described in Example 2 below. The third mode is preferred, since this provides anti-caries benefits, whilst leaving the natural oral cavity microflora undisturbed. Thus, preferably, the bacterial production of acid can be reduced or eliminated without significantly disturbing the oral cavity's natural microflora. This may be achieved by inhibiting the bacteria responsible for the production of acid, in particular Streptococcus mutans, at a concentration below the MIC.

In an even further aspect the present invention provides use of one or more of the flavour materials of the flavour composition of the invention, for the purpose of reducing and/or preventing dental caries.

The flavour composition typically also includes other flavour ingredients (which may be selected from the 400-500 or so flavour materials that are in current use when formulating flavour compositions) chosen to give desired overall flavour characteristics to the composition.

The flavour composition of the invention can be readily made by simply mixing the specified ingredients, as is well known to those skilled in the art.

The flavour compositions of the invention find application in a wide range of consumer products, particularly oral care products such as toothpastes, mouthwashes, chewing gum (where the term “chewing gum” is intended also to encompass bubble gum), confectionery, dental floss, dissolvable mouth films, breath sprays and breath freshening tablets.

The present invention also includes within its scope consumer products, particularly oral care products, including a flavour composition in accordance with the invention.

The consumer products, particularly oral care products, which include a flavour composition in accordance with the invention may be formulated in a conventional manner as is well known to those skilled in the art. For example, a toothpaste formulation will typically include from 0.3% to 2.0% by weight, preferably from 0.5% to 1.5% by weight, and more preferably from 0.8% to 1.2% by weight, of the flavour composition. A mouthwash will typically contain the flavour composition in an amount in the range 0.05% to 2.0% by weight, preferably from 0.1% to 1.0% by weight, and more preferably from 0.15% to 0.5% by weight. For a chewing gum, the composition of the invention may be present in an amount in the range 0.5% to 3.5% by weight, preferably from 0.75% to 2.0% by weight, and more preferably from 1.0% to 1.75% by weight.

A consumer product may conveniently also include ingredients such as fluoride, zinc salts, pyrophosphates etc, known to have an effect in reducing and/or preventing dental caries. These ingredients can be present in lower amounts than is typically conventional.

In an even further aspect, the present invention provides a consumer product comprising a flavour composition in accordance with the invention; and a fluoride-ion providing compound.

The fluoride-ion providing compound is capable of releasing fluoride ions or fluoride-containing ions in water. Suitable fluoride-ion providing compounds include, for example, sodium fluoride, potassium fluoride, ammonium fluoride, cuprous fluoride, zinc fluoride, stannic fluoride, stannous fluoride, barium fluoride, sodium fluorosilicate, ammonium fluorosilicate, sodium fluorozirconate, sodium monofluorophosphate, aluminium mono- and difluorophosphate and fluorinated sodium calcium pyrophosphate.

The invention also covers a consumer product comprising a flavour composition in accordance with the invention and xylitol.

It has been found by the present inventors that the combination of xylitol and a flavour composition of the invention can produce a synergistic effect, with the xylitol and flavour composition giving a greater combined effect in reducing the production of acid by Streptococcus mutans than xylitol and flavour composition alone. The potential synergy between xylitol and a flavour composition in accordance with the invention can be investigated using the method described in Example 4 below.

The invention will be illustrated by the following examples.

### EXAMPLE 1(a)

### Microtitre Total Acid Inhibition (TAI) Test

The following method was used to determine the efficacy of a flavour material or flavour composition at inhibiting acid production by the micro-organism Streptococcus mutans.

250 ml of PM broth (containing: peptone, 2% w/v; tryptone, 1% w/v; yeast extract, 1% w/v; KCl, 0.25% w/v; of approximately pH 7) was charged to a Duran bottle bunged with a breathable stopper and inoculated with the test strain Streptococcus mutans R9, deposited under the Budapest Treaty with National Collections of Industrial, Food and Marine Bacteria Limited, 23 St Machar Drive, Aberdeen, AB24 3RY, Scotland, UK on 22 Jan. 2004 and given accession number NCIMB 41209 (may also be obtained from Prof. Philip Marsh, Centre for Applied Microbiology and Research, Salisbury, Wiltshire, SP4 0JG, UK). The bacterial culture was incubated anaerobically at 37° C. for 48 hours. The optical density of the culture at 540 nm (OD₅₄₀) was measured and adjusted (if required) to between 0.2 and 0.3, by diluting with fresh PM broth to give a stock inoculum culture.

Acid indicator broth (AIB) was prepared by adding 4% (w/v) glucose and 0.8% (v/v) of a Bromocresol Purple stock solution (stock solution contains 16 g Bromocresol Purple in 1000 ml ethanol) to 0.3% (w/v) TSB broth (tryptone soya broth, available from Oxoid, Basingstoke, UK). The resulting AIB was sterilised by aseptically passing the solution through a 0.22 μm filter into a sterile bottle.

Stock solutions of flavour material(s) or flavour composition(s) (flavour(s)) were made to 10,000 ppm by adding 50 mg of neat flavour material/flavour to 5 ml of AIB, and vigorously mixing the mixture by vortex. Each row of a standard, 96-well plastic microtitre plate (labelled A-H) was allocated to one sample, thus eight samples per plate. Row H contained only Schaedler broth for use as a bacterial control to indicate the degree of turbidity resulting from bacterial growth in the absence of any test material. Aseptically, 200 μl of the initial dilution of flavour material/flavour was transferred to the 1^{st} and 7^{th} well of the appropriate row. All other test wells were filled with 100 μl of sterile Schaedler broth using an 8-channel micro-pipette. The contents of each of the wells in column 1 were mixed by sucking samples up and down in pipette tips, before 100 μl was transferred to column 2. The same sterile pipette tips were used to transfer 100 μl of each well in column 7, into the appropriate well in column 8. This set of eight tips was then discarded into disinfectant solution. Using eight fresh, sterile tips the process was repeated by transferring 100 μl from column 2 into column 3 (and 8 into 9). The process was continued until all wells in columns 6 and 12 contained 200 μl. After mixing, 100 μl was discarded from each of the wells in columns 6 and 12 to waste. Finally, 100 μl of the bacterial stock inoculum culture was added to all wells (except the control, no bacteria wells in row H), thus giving a final volume of 200 μl in each well. The final concentration of ingredients was thus 5,000 ppm in columns 1 and 7; 2,500 ppm in columns 2 and 8; and so forth so that the final concentration of ingredients in columns 6 and 12 was 156 ppm.

The plates were incubated anaerobically (80% N₂, 10% H₂, 10% CO₂) for 24 hours at 37° C. Following incubation, the plates were read by eye. If the wells of bacteria/broth remained purple then the flavour material/flavour had successfully inhibited lactic acid production by S. mutans. If the wells of bacteria/broth appeared yellow, then S. mutans had metabolised glucose to lactic acid and the flavour material/flavour had not inhibited acid production.

Results were recorded as the lowest concentration at which the flavour material/flavour inhibited acid production.

### EXAMPLE 1(b)

### Bottle Total Acid Inhibition (TAI) Test

250 ml of PM broth (of formulation as described in Example 1(a) above) was charged to a Duran bottle bunged with a breathable stopper and inoculated with a loopful of Streptococcus mutans R9 (as above). The bacterial culture was then incubated anaerobically for 2-3 days at 37° C., followed by centrifugation at 3600 rpm for 10 minutes. The supernatant was decanted to waste. The pellets remaining were resuspended in 12 ml of 0.1% peptone and the optical density at 540 nm (OD₅₄₀) measured and adjusted (if required) by diluting with fresh PM broth to between 0.2 and 0.3 to give a stock inoculum culture.

Broth was prepared by adding 4% (w/v) glucose to 0.3% (w/v) TSB broth (GTSB). The broth was sterilised by aseptically passing the solution through a 0.22 μm filter into a sterile bottle.

Control incubations were prepared by adding 2.5 μl of the stock inoculum culture (adjusted to an OD₅₄₀ of 0.2-0.3) to 2.5 ml of GTSB containing 4% (w/v) glucose in 0.3% (w/v) TSB.

A test flavour material/flavour was diluted in the GTSB to give a stock solution of flavour material/flavour with a final concentration of 25,000 ppm (250 mg flavour material/flavour in 10 ml of GTSB).

Flavour material/flavour incubations were prepared by adding 2.5 ml of the stock inoculum culture to 2.45 ml of GTSB, and 50 μl of flavour material/flavour stock solution. Thus, flavour materials/flavours were tested at a final concentration of 250 ppm, for their efficacy in inhibiting acid production from 2% glucose.

The mixtures were then incubated anaerobically.

After anaerobic incubation of the resulting mixtures for 18-24 hours, the pH of the suspensions was measured using a 476530M combination pH electrode (Mettler Toledo, 64 Boston Road, Beaumont Leys, Leicester, LE4 1AW), calibrated using pH 4 and pH 7 buffers. Results were recorded as the difference in pH change between broths containing flavour material/flavour and an untreated control.

### EXAMPLE 2

### Minimum Inhibitory Concentration (MIC)

The minimum inhibitory concentration of a flavour material or flavour composition (flavour) was determined by the following method.

A culture of the test strain Streptococcus mutans R9, deposited under the Budapest Treaty with National Collections of Industrial, Food and Marine Bacteria Limited, 23 St Machar Drive, Aberdeen, AB24 3RY, Scotland, UK on 22 Jan. 2004 and given accession number NCIMB 41209 (may also be obtained from Prof. Philip Marsh, Centre for Applied Microbiology and Research, Salisbury, Wiltshire, SP4 0JG, UK) was grown in 250 ml of PM broth (containing: peptone, 2% w/v; tryptone, 1% w/v; yeast extract, 1% w/v; KCl, 0.25% w/v; of approximately pH 7), anaerobically at 37° C. for 48 hours. The optical density of the culture at 540 nm (OD₅₄₀) was measured and adjusted to 0.2-0.3 by diluting with fresh PM broth. The culture was then diluted in Schaedler broth (Oxoid, Basingstoke, UK) in a ratio of 1 part culture to 25 parts broth to give a stock inoculum culture.

Flavour or flavour materials were diluted in sterile Schaedler broth to yield a 10,000 ppm stock solution, and the mixture vigorously mixed by vortex. Each row of a standard, 96-well plastic microtitre plate (labelled A-H) was allocated to one sample, thus eight samples per plate. Row H contained only Schaedler broth for use as a bacterial control to indicate the degree of turbidity resulting from bacterial growth in the absence of any test material. Aseptically, 200 μl of the initial dilution of flavour/flavour material was transferred to the 1^{st} and 7^{th} well of the appropriate row. All other test wells were filled with 100 μl of sterile Schaedler broth using an 8-channel micro-pipette. The contents of each of the wells in column 1 were mixed by sucking samples up and down in pipette tips, before 100 μl was transferred to column 2. The same sterile pipette tips were used to transfer 100 μl of each well in column 7, into the appropriate well in column 8. This set of eight tips was then discarded into disinfectant solution. Using eight fresh sterile tips the process was repeated by transferring 100 μl from column 2 into column 3 (and 8 into 9). The process was continued until all wells in columns 6 and 12 contained 200 μl. After mixing, 100 μl was discarded from each of the wells in columns 6 and 12 to waste. Finally, 100 μl of the pre-diluted stock inoculum culture was added to all wells (except the control, no bacteria wells in row H), thus giving a final volume of 200 μl in each well.

A blank plate was prepared for each set of eight samples by repeating the process described above, except that 100 μl of Schaedler broth was added instead of bacterial culture. This plate was used as the control plate against which the test plate(s) could be read.

Test and control plates were sealed using autoclave tape and incubated for 48 hours anaerobically at 37° C.

A microtitre plate reader (Model MRX, Dynatech Laboratories) was preset to gently agitate the plates and mix the contents. The absorbance at 540 nm “A₅₄₀” was used as a measure of turbidity resulting from bacterial growth. The control, un-inoculated plate for each set of samples was read first, and the plate reader then programmed to use the control readings to blank all other plate readings for the inoculated plates for the same set of test materials (i.e. removing turbidity due to flavour and possible colour changes during incubation). Thus, the corrected readings generated were absorbances resulting from turbidity from bacterial growth. The MIC was taken as the concentration of flavour/flavour material required to inhibit growth so that the change in absorbance during the incubation period was <0.2 A₅₄₀.

### EXAMPLE 3

Flavour materials useful in a flavour composition of the invention were tested at 250 ppm for their potential synergy with fluoride as described below.

250 ml of PM broth (of formulation as described in Example 1(a) above) was charged to a Duran bottle bunged with a breathable stopper and inoculated with a loopful of Streptococcus mutans R9 (as above). The bacterial culture was then incubated anaerobically for 2-3 days at 37° C., followed by centrifugation at 3600 rpm for 10 minutes. The supernatant was decanted to waste. The pellets remaining were resuspended in 12 ml of 0.1% peptone and the optical density at 540 nm (OD₅₄₀) measured and adjusted (if required) by diluting with fresh PM broth to between 0.2 and 0.3 to give a stock inoculum culture.

Broth was prepared by adding 4% (w/v) glucose to 0.3% (w/v) TSB broth (GTSB). The broth was sterilised by aseptically passing the solution through a 0.22 μm filter into a sterile bottle.

Control and flavour material incubations were prepared as described in Example 1(b) above.

A fluoride stock solution (2,500 ppm F—) was prepared by dissolving 0.552 g NaF in 10 ml of GTSB. For fluoride controls, 2.5 ml of stock inoculum culture was added to 2.48 ml of GTSB, and 20 μl of fluoride stock solution.

For flavour material and fluoride incubations, 2.5 ml of stock inoculum culture was added to 2.43 ml of GTSB, 50 μl of flavour material stock solution and 20 μl of fluoride stock solution.

The mixtures were incubated anaerobically.

After anaerobic incubation of the resulting mixtures for 18-24 hours, the pH of the suspensions was measured using a 476530M combination pH electrode (Mettler Toledo, 64 Boston Road, Beaumont Leys, Leicester, LE4 1AW), calibrated using pH 4 and pH 7 buffers. Results were recorded as the difference in pH change between broths.

If the pH of the incubated broth containing flavour material and fluoride was higher than that measured for incubated broths containing either flavour material or fluoride, then this was considered to indicate that there had been a synergistic effect between the flavour material and fluoride in reducing the acid production of Streptococcus mutans.

The results are presented below, where

++++=Inhibition of acid production by an additional 0.75 pH units or more (i.e.
>0.75 pH units in addition to the effect of fluoride or flavour material alone);
+++=Inhibition of acid production by an additional 0.50-0.74 pH units;
++=Inhibition of acid production by an additional 0.25-0.49 pH units; and
+=Inhibition of acid production by an additional 0.01-0.24 pH units.

| Flavour Material (at 250 ppm) | Synergy with Fluoride |
|---|---|
| Alcohol C10 (Decanol) | ++++ |
| Aldehyde C10 (Decanal) | ++ |
| Anethole Synthetic | ++++ |
| Basil Comores | ++++ |
| Cinnamic Aldehyde Extra | ++ |
| Citral Natural | + |
| Origanum | ++ |
| Peppermint Arvensis Terpeneless ACF 153 | + |
| Peppermint Chinese Triple Rectified (Quest) | ++/+++ |
| Clove Bud Rectified Extra | ++ |
| Ginger | + |
| Peppermint American Willamette Natural | ++++ |
| Peppermint Indian Rectified | + |
| Tea Tree Pure | + |
| Thymol | + |
| Cardamom English Distilled | ++++ |
| Damascone F | +++ |
| Ionone Alpha | ++/+++ |
| Tarragon | + |

### EXAMPLE 4

Xylitol is a sugar substitute that has been used in many products as a non-cariogenic sweetener. The potential synergy between xylitol and a flavour material useful in the composition of the invention was investigated using a glucose/xylitol broth by the following method. Flavour materials were tested at 250 ppm unless otherwise stated.

250 ml of PM broth (of formulation as described in Example 1(a) above) was charged to a Duran bottle bunged with a breathable stopper and inoculated with a loopful of Streptococcus mutans R9 (as above). The bacterial culture was then incubated anaerobically for 2-3 days at 37° C., followed by centrifugation at 3600 rpm for 10 minutes. The supernatant was decanted to waste. The pellets remaining were resuspended in 12 ml of 0.1% peptone and the optical density at 540 nm (OD₅₄₀) measured and adjusted (if required) by diluting with fresh PM broth to between 0.2 and 0.3 to give a stock inoculum culture.

Broth was prepared by adding 4% (w/v) glucose to 0.3% (w/v) TSB broth (GTSB). The broth was sterilised by aseptically passing the solution through a 0.22 μm filter into a sterile bottle.

Control and flavour material incubations were prepared as described in Example 1(b) above. Xylitol synergy was investigated by adding 2.5 ml of stock inoculum culture to 2.5 ml of GTSB supplemented with 4% (w/v) xylitol. An additional control was also prepared with 2.5 ml of stock inoculum culture added to 2.5 ml of a 4% (w/v) xylitol solution in 0.3% TSB (no glucose).

These mixtures were incubated anaerobically.

After anaerobic incubation of the resulting mixtures for 18-24 hours, the pH of the suspensions was measured using a 476530M combination pH electrode (Mettler Toledo, 64 Boston Road, Beaumont Leys, Leicester, LE4 1AW), calibrated using pH 4 and pH 7 buffers. Results were recorded as the difference in pH change between broths.

If the pH of the incubated broth containing flavour material and xylitol was higher than that recorded for incubated broths containing either flavour material or xylitol, then this was considered to indicate that there was a synergy between the flavour material and xylitol in reducing the acid production of Streptococcus mutans.

The results for some flavour materials useful in a composition of the invention are presented below, where the degrees of synergy were allocated as described in Example 3.

| Flavour Material (at 250 ppm unless stated) | Synergy with Xylitol |
|---|---|
| Ginger | ++++ |
| Ionone Alpha (125 ppm) | ++++ |
| Alcohol C9 (62.5 ppm) | +++ |
| Basil Comores | ++ |
| Damascone | ++ |
| Aldehyde C9 | +/++ |
| Origanum (125 ppm) | +/++ |
| Aniseed rectified | + |
| Peppermint Aspen | + |
| Peppermint Moroccan | + |

### EXAMPLE 5

A flavour composition in accordance with the invention was prepared by mixing the following ingredients:

| Ingredient | % w/w | Group |
|---|---|---|
| C9 Aldehyde (nonanal) | 0.1 | (b) |
| Anethole Synthetic | 9.0 | (a) |
| Cis 3 Hexenyl Butyrate | 2.0 | |
| Menthol Laevo | 45.0 | |
| Orange Oil | 4.5 | |
| Origanum | 0.9 | (a) |
| Peppermint American Yakima Rectified | 31.5 | (b) |
| Peppermint Arvensis Terpeneless | 7.0 | (b) |
| Total | 100 | |

### EXAMPLE 6

A flavour composition in accordance with the invention was prepared by mixing the following ingredients:

| Ingredient | % w/w | Group |
|---|---|---|
| Alcohol C9 (nonanol) | 0.15 | (a) |
| Anethole Synthetic | 8.50 | (a) |
| Cinnamic Aldehyde | 2.25 | (a) |
| Citral | 6.60 | (b) |
| Menthol laevo | 42.50 | |
| Orange Oil | 4.25 | |
| Peppermint American Yakima Rectified | 29.75 | (b) |
| Peppermint Arvensis Terpeneless | 6.00 | (b) |
| Total | 100 | |

### EXAMPLE 7

A flavour composition in accordance with the invention was prepared by mixing the following ingredients:

| Ingredient | % w/w | Group |
|---|---|---|
| Anethole Synthetic | 7.0 | (a) |
| Clove Bud Oil Rectified | 6.0 | (b) |
| Menthol laevo | 35.0 | |
| Orange Oil | 3.5 | |
| Peppermint American Yakima Rectified | 36.4 | (b) |
| Peppermint Chinese Triple Rectified | 12.0 | (b) |
| Origanum | 0.1 | (a) |
| Total | 100 | |

### EXAMPLE 8

### Formulations

Any one of the flavour compositions of Examples 5 to 7 above may be included in the following toothpaste, mouthwash, or chewing gum formulations, which are prepared according to conventional methods known to those skilled in the art:

Chalk Toothpaste

| Material | % w/w |
|---|---|
| Glycerine | 20.0 |
| Distilled Water | 35.3 |
| Calcium Carbonate (Sturcal H) | 40.0 |
| Sodium Carrageenate (Viscarin) | 2.00 |
| Sodium Saccharin | 0.20 |
| Sodium Lauryl Sulphate (Empicol LZPV/C) | 1.50 |
| Flavour Composition | 1.00 |
| Total | 100.00 |

where Sturcal H, Viscarin and Empicol LZPV/C are all Trade Marks.

Opacified Silica Toothpaste

| Material | % w/w |
|---|---|
| Sorbitol 70% syrup | 50.0 |
| Distilled Water | 23.6 |
| Sodium Monofluorophosphate | 0.80 |
| Trisodium Phosphate 12H₂O | 0.10 |
| Sodium Saccharin | 0.20 |
| Precipitated Silica (AC 30) | 8.00 |
| Precipitated Silica (TC 15) | 8.00 |
| Sodium Carboxy Methyl Cellulose (9M31XF) | 0.80 |
| Titanium Dioxide (Tiona) | 1.00 |
| Sodium Lauryl Sulphate (Empicol LZPV/C) | 1.50 |
| Polyethylene Glycol 1500 | 5.00 |
| Flavour Composition | 1.00 |
| Total | 100.00 |

### Where Tiona and Empicol LZPV/C are Trade Marks.

### Ready-to-Use Mouthwash

% w/w

Mixture A - Alcohol Phase

| | |
|---|---|
| Ethanol 96%, Double Rectified | 12.000 |
| PEG 40 Hydrogenated Castor Oil (Cremophor RH40) | 0.250 |
| Flavour Composition | 0.200 |

Mixture B - Aqueous Phase

| | |
|---|---|
| Sorbitol 70% syrup | 12.000 |
| Saccharin 25% solution | 0.200 |
| Cetyl Pyridinium Chloride | 0.025 |
| Distilled Water | 75.325 |

### Where Cremophor RH40 is a Trade Mark.

The alcohol phase (mixture A) and aqueous phase (mixture B) were prepared separately and then combined to give the mouthwash.

Chewing Gum

| Material | % w/w |
|---|---|
| Gum Base Balear T | 28.0 |
| Sorbitol Powder | 52.9 |
| Sorbitol Syrup | 6.0 |
| Xylitol | 6.0 |
| Glycerol 98% | 5.0 |
| Aspartame | 0.05 |
| Acesulfame K | 0.05 |
| Flavour Composition | 2.0 |

where Balear T and Acesulfame K are Trade Marks.

1.-10. (canceled)

11. A method for reducing or preventing acid-producing bacteria in the mouth which comprises introducing into the mouth an effective amount of an oral care product comprising a flavour composition comprising at least two flavour materials selected from the following Group (a) materials: decanol, nonanol, decanal, anethole synthetic, cardamom oil, cinnamic aldehyde, ionone alpha, origanum, tarragon, thymol; and at least two flavour materials selected from the following Group (b) materials: nonanal, Aniseed rectified, basil oil, camomile oil, citral, clove bud oil, Damascone F, ginger, Tea Tree Pure, peppermint oil of natural origin, said flavour composition containing at least 6% by weight of flavour materials from Group (a).

12. A method for inhibiting the production of acid from the metabolism of dietary sugar by micro-organisms present in the oral cavity which comprises orally administering to a host in need of such inhibition, an oral care product comprising a flavour composition comprising at least two flavour materials selected from the following Group (a) materials: decanol, nonanol, decanal, anethole synthetic, cardamom oil, cinnamic aldehyde, ionone alpha, origanum, tarragon, thymol; and at least two flavour materials selected from the following Group (b) materials: nonanal, Aniseed rectified, basil oil, camomile oil, citral, clove bud oil, Damascone F, ginger, Tea Tree Pure, peppermint oil of natural origin, said flavour composition containing at least 6% by weight of flavour materials from Group (a).

13. The method of claim 11 wherein the oral care product is administered to reduce the production of acid by Streptococcus mutals.

14. A method of claim 11 wherein the flavour composition comprises at least three flavour materials from Group (a) at least 10% by weight of flavour materials from Group (a) and at least 10% by weight of flavour materials from Group (b).

15. The method of claim 14 wherein the oral care product also comprises a fluoride-ion providing compound.

16. The method of claim 11 wherein the oral care product also includes xylitol.
